(19) United States
(12) Patent Application Publication
(10) Pub. No.: US 2010/0226886 A1
(43) Pub. Date: Sep. 9, 2010

(54) Combinations of MTP Inhibitors with Cholesterol Absorption Inhibitors or Interferon for Treating Hepatitis C

(76) Inventor: Willliam J. Sasiela, Bridgewater, NJ (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/572,860

(22) Filed: Oct. 2, 2009

Related U.S. Application Data
(63) Continuation of application No. PCT/US08/58992, filed on Apr. 1, 2008.
(60) Provisional application No. 60/909,833, filed on Apr. 3, 2007.

(51) Int. Cl. A61K 38/21; A61K 39/395; A61K 39/29; A61K 31/7088; A61K 31/437; A61K 31/4439; A61K 31/455; A61K 31/426; A61K 31/13

(52) U.S. Cl. 424/85.7; 424/133.1; 424/228.1; 514/44 A; 514/299; 514/342; 514/356; 514/371; 514/662

(57) ABSTRACT

The invention is directed in part to methods for treating and/or controlling hepatitis C in a patient. The methods are directed in part to combination therapies using a microsomal triglyceride transfer protein (MTP) inhibitor (for example, AEGR-733 and implitapide) and at least one other active agent.

### RELATED APPLICATIONS

This application claims priority to U.S. provisional patent application U.S. Ser. No. 60/909,833, filed Apr. 3, 2007, hereby incorporated by reference in its entirety.

### FIELD OF THE INVENTION

This invention relates generally to methods of treating and/or controlling hepatitis C in a patient. More particularly, the invention relates to therapies using a microsomal triglyceride transfer protein (MTP) inhibitor in combination with at least one other active agent.

### BACKGROUND

Hepatitis C(HCV) is an infectious viral disease caused by a small (50-60 nm), enveloped, positive, single-stranded RNA virus in the Flaviviridae family. The virus mutates rapidly, and has extensive genetic heterogeneity, with at least six different genotypes and more than 90 subtypes. An estimated 150-200 million people worldwide are infected with HCV.

Unlike hepatitis A, which is caused by a picornavirus and is transmitted through oral-fecal contact with contaminated food, HCV is spread by blood-to-blood contact with an infected person's blood. Currently, vaccines are only available for hepatitis A and hepatitis B.

Acute HCV is often asymptomatic and about 70-80% of patients infected with HCV develop chronic HCV. If left untreated, chronic HCV may lead to cirrhosis and/or liver cancer. HCV is the leading cause of liver transplants in the United States.

A diagnosis of hepatitis C is rarely made during the acute phase of the disease because the majority of people infected experience no symptoms during this phase of the disease. The diagnosis of chronic phase hepatitis C is also challenging due to the absence or lack of specificity of symptoms until advanced liver disease develops, which may not occur until decades into the disease.

Hepatitis C testing usually includes serological blood tests used to detect antibodies to HCV. Anti-HCV antibodies can be detected in 80% of patients within 15 weeks after exposure, in >90% within 5 months after exposure, and in >97% by 6 months after exposure. The presence of the virus can be tested using molecular nucleic acid testing methods such as polymerase chain reaction (PCR), transcription mediated amplification (TMA), or branched DNA (b-DNA). Most HCV nucleic acid molecular tests have the capacity to detect not only whether the virus is present, but also to measure the amount of virus present in the blood (the HCV viral load). Tests that detect antibodies against the virus include the enzyme immunoassay (EIA) which contains HCV antigens from the virus core and nonstructural genes, and the recombinant immunoblot assay. The National Institutes of Health recommend HCV infection in a patient with a positive EIA test should be confirmed by a qualitative HCV RNA assay with a lower limit detection of 50 IU/ml or less.

Several formulations of recombinant alpha interferon are available as therapy of hepatitis C. Alpha interferon is a host protein that is made in response to viral infections and has natural antiviral activity. Interferon therapies also now include polyethylene glycol modified, or pegylated, interferons.

Currently the standard therapy for hepatitis C infection is a 24- or 48-week course of the combination of pegylated alpha interferon and ribavirin, an oral antiviral agent that has activity against a broad range of viruses. By itself, ribavirin has little effect on HCV, but adding it to interferon or peginterferon increases the sustained response rate by two- to three-fold. The optimal duration of treatment depends on viral genotype. Patients with genotypes 2 and 3 have a high rate of response to interferon and ribavirin treatment (70 to 80 percent). In contrast, patients with genotype 1 have a lower rate of response to such therapy (40 to 45 percent).

Alpha interferon has multiple neuropsychiatric effects, can induce an autoimmune condition, and strict abstinence from alcohol is also recommended during therapy with interferon. Prolonged therapy can cause marked irritability, anxiety, personality changes, depression and even suicide or acute psychosis. Ribavirin can cause red cell haemolysis to a variable degree in almost all patients, and patients with a pre-existing haemolysis or anaemia should not receive ribavirin. Fatal myocardial infarctions and strokes have been reported during combination therapy with alpha interferon and ribavirin. Ribavirin also causes birth defects in animal studies, while alpha interferon has direct antigrowth and antiproliferative effects.

Few options exist for patients who either do not respond to therapy or who respond and later relapse. Further, it is well known that drug-resistant viruses emerge after the introduction of anti-viral drugs.

Microsomal triglyceride transfer protein (MTP) inhibitors have been developed as potent inhibitors of MTP-mediated neutral lipid transfer activity. One exemplary MTP inhibitor is BMS-201038, developed by Bristol-Myers Squibb. See, U.S. Pat. Nos. 5,739,135; and 5,712,279. Studies using an animal model for homozygous familial hypercholesterolemia indicated that BMS-201038 effectively reduced plasma cholesterol levels in a dose dependent manner, for example, at 25 mg/day, suggesting that this compound might be effective for treating patients with hypercholesterolemia. It was noticed, however, that certain patients treated with 25 mg/day of BMS-201038 experienced certain adverse events, for example, gastrointestinal disturbances, abnormalities in liver function, and hepatic steatosis. Another potent MTP inhibitor known as implitapide has been developed. See, U.S. Pat. Nos. 6,265,431, 6,479,503, 5,952,498. During clinical studies, dosages of implitapide of 80 mg/day or greater, although therapeutically effective for hypercholesterolemia, were found to result in certain adverse events, for example, gastrointestinal disturbances, abnormalities in liver function, and hepatic steatosis.

A link between HCV production and VLDL assembly has been recently identified. (Huang et al., PNAS, v. 104, 5848-5853 (2007). At least a portion of hepatitis C virus secreted by hepatocytes circulates in the blood as a complex with very low density lipoproteins (VLDLs). Huang et al. observed that HCV replication complexes colocalized with apolipoprotein B (apoB), apolipoprotein E (apoE), and microsomal triglyceride transfer protein (MTP), suggesting that HCV RNA replication occurs in a cellular compartment mediating VLDL assembly. To determine if VLDL assembly and secretion are required for HCV replication, Huang et al. treated cells containing HCV replicons with a MTP inhibitor. Huang et al. observed that treating the cells with a MTP inhibitor reduced the amount of HCV RNA in the medium and the viral titer by approximately 80%, suggesting that VLDL secretion may be required for the cellular release of HCV particles.

There is a clear on-going need for methods for treating hepatitis C that reduce treatment times, are efficacious for e.g. drug-resistant viruses and/or have fewer side effects.

### SUMMARY OF THE INVENTION

The invention provides methods for treating acute and/or chronic hepatitis C. The method includes administering specific MTP inhibitors, such as implitapide, and/or administering MTP inhibitors, such as AEGR-733 or implitapide, in combination with at least one other active agent, for example, a cholesterol, lipid, or lipoprotein lowering or inhibition agent, for example ezetimibe, and/or a hepatitis C treatment agent, for example, interferon or pegylated interferon, optionally together with ribavirin. The MTP inhibitors can be administered at certain lower dosages that are still therapeutically effective but create fewer or reduced adverse effects when compared to higher dosage therapies.

In one aspect, a method of treating a hepatitis C infection is provided, comprising administering to a patient in need thereof: i) a MTP inhibitor and ii) at least one other active agent. The at least one other active agent may be a hepatitis C treatment agent and/or a cholesterol lowering agent. One, two or more other active agents are contemplated. The patient may be for example a human.

The disclosed methods contemplate that the MTP inhibitor and the other active agent can be administered sequentially, or substantially simultaneously.

The MTP inhibitor and the other active agent can be administered in separate dosage forms, or as a single dosage form. In some embodiments, administering a MTP inhibitor in combination with the other active agent provides a synergistic therapeutic effect. The MTP inhibitor and/or the other active agent(s) can be administered in a synergistically effective amount.

The administration of one or more MTP inhibitors, when administered with at least one other active agent, may provide an additive or synergistic therapeutic effect, e.g. may result in a greater reduction in the hepatitis C viral load of a patient that is greater than the sum of the hepatitis C viral load reduction upon administration of a MTP inhibitor alone and an other active agent alone. The viral load may be for example, measured using molecular nucleic acid testing methods. The viral load can measured at about 14 days, 28 days, 4 months and/or 6 months from a first administration of the MTP inhibitor and/or the one or more other active agents

A method of treating hepatitis C is also disclosed that comprises administering to a patient in need thereof a MTP inhibitor in combination with at least one additional active agent, wherein the administration of the combination results a shorter treatment duration as compared to administration of the additional active agent or the MTP inhibitor alone.

In another embodiment, a method of treating hepatitis C is provided that comprises administering to a patient in need thereof a MTP inhibitor in combination with at least one additional active agent, wherein the patient is less likely to have a sustained virological response as compared to administration of the additional active agent or the MTP inhibitor alone.

One contemplated method of treating a hepatitis C infection includes administering to a patient in need thereof: i) implitapide and optionally, ii) at least one other active agent.

Compositions and kits for use in the treatment of hepatitis are also disclosed. Such compositions and kits comprise a MTP inhibitor and a hepatitis C treatment agent.

The combination treatments disclosed herein may also lead less viral resistance as compared to traditional treatments, e.g. alpha-inteferon alone or in combination with ribavirin. In other embodiments, the combination treatments disclosed herein may have a significant reduction in treatment duration.

### DETAILED DESCRIPTION

The invention relates, in part, to methods of treating hepatitis C comprising administering to a patient in need thereof a MTP inhibitor in combination with at least one other active agent. Such a patient may be suffering from, e.g. chronic hepatitis C and/or may have a viral load of greater than about 2 million virons/ml.

The methods described herein may result in a lower viral load after, for example, twelve, twenty-four, forty-eight and/or fifty-two weeks of daily administration as compared to daily administration of one or more other active agents, or a MTP inhibitor alone, for the same time interval.

Administering combinations of a MTP inhibitor and another active agent, under certain circumstances, provide an additive and/or synergistic therapeutic effect, e.g. provide a reduction hepatitis C viral load that is greater than the sum of the reduction in viral load resulting from a) administering a MTP inhibitor alone, or b) administering one or more other active agents alone.

### DEFINITIONS

For convenience, certain terms used in the specification, examples, and appended claims are collected in this section.

The phrase “combination therapy,” as used herein, refers to co-administering an MTP inhibitor, for example, AEGR-733 and implitapide, or a combination thereof, and another active agent, for example, a cholesterol, lipid, or lipoprotein lowering or inhibition agent, and/or a hepatitis C treatment agent, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules or tablets for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

Combination therapy can also embrace the administration of the therapeutic agents as described above in further combination with other biologically active ingredients and non-drug therapies. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The components of the combination may be administered to a patient simultaneously or sequentially. It will be appreciated that the components may be present in the same pharmaceutically acceptable carrier and, therefore, can be administered simultaneously. Alternatively, the active ingredients may be present in separate pharmaceutical carriers, such as conventional oral dosage forms, that can be administered either simultaneously or sequentially.

The terms, “individual,” “patient,” or “subject” are used interchangeably herein and include any mammal, including animals, for example, primates, for example, humans, and other animals, for example, dogs, cats, swine, cattle, sheep, and horses. The methods of the invention can be practiced on a mammal, such as a human, but can also be other mammals, for example, an animal in need of veterinary treatment, for example, domestic animals (for example, dogs, cats, and the like), farm animals (for example, cows, sheep, pigs, horses, and the like) and laboratory animals (for example, rats, mice, guinea pigs, and the like).

The phrase “minimizing adverse effects,” “reducing adverse events,” or “reduced adverse events,” as used herein refer to an amelioration or elimination of one or more undesired side effects associated with, e.g. the use of MTP inhibitors in the present invention. Side effects of traditional use of the MTP inhibitors include, without limitation, diarrhea, nausea, gastrointestional disorders, steatorrhea, abdominal cramping, distention, elevated liver function tests, fatty liver (hepatic steatosis); hepatic fat build up, polyneuropathy, peripheral neuropathy, rhabdomyolysis, arthralgia, myalgia, chest pain, rhinitis, dizziness, arthritis, peripheral edema, gastroenteritis, liver function tests abnormal, colitis, rectal hemorrhage, esophagitis, eructation, stomatitis, biliary pain, cheilitis, duodenal ulcer, dysphagia, enteritis, melena, gum hemorrhage, stomach ulcer, tenesmus, ulcerative stomatitis, hepatitis, pancreatitis, cholestatic jaundice, paresthesia, amnesia, libido decreased, emotional lability, incoordination, torticollis, facial paralysis, hyperkinesia, depression, hypesthesia, hypertonia, leg cramps, bursitis, tenosynovitis, myasthenia, tendinous contracture, myositis, hyperglycemia, creatine phosphokinase increased, gout, weight gain, hypoglycemia, anaphylaxis, angioneurotic edema, and bullous rashes (including erythema multiforme, Stevens-Johnson syndrome, and toxic epidermal necrolysis). Accordingly, the methods described herein may provide an effective therapy while at the same time may cause fewer or less significant adverse events as compared to larger monotherapies alone.

In certain embodiments, the doses and/or dosage forms of MTP inhibitors provided herein at least partially eliminates such side effects. As used herein, the phrase “partially eliminated” refers to a reduction in the severity, extent, or duration of the particular side effect by at least 30%, 40%, 50%, 60%, 70%, 80%, 90%, 95% and 99% relative to that found by administering 25 mg/day of AEGR-733 during monotherapy or either 80 mg/day or 160 mg/day of implitapide during monotherapy. In certain embodiments, substantially all side effects are not manifested. Those skilled in the art are credited with the ability to detect and grade the severity, extent, or duration of side effects as well as the degree of amelioration of a side effect. In some embodiments, two or more side effects are ameliorated.

The term “synergistic” refers to two or more agents, e.g. a MTP inhibitor and another active agent, that when taken together, produce a total joint effect that is greater than the sum of the effects of each drug when taken alone.

The term, “therapeutically effective” refers to the ability of an active ingredient, alone or in combination with another active agent, to elicit the biological or medical response that is being sought by a researcher, veterinarian, medical doctor or other clinician.

The term, “therapeutically effective amount” includes the amount of an active ingredient, or combination of active ingredients, that will elicit the biological or medical response that is being sought by the researcher, veterinarian, medical doctor or other clinician. The active agents of the invention are administered in amounts effective at lowering the hepatitis C viral load in a patient. Alternatively, a therapeutically effective amount of an active ingredient is the quantity of the compound or agent required to achieve a desired therapeutic and/or prophylactic effect, such as the amount of the active agent that results in the prevention of or a decrease in the symptoms associated with the condition (for example, to meet an end-point).

The terms, “pharmaceutically acceptable” or “pharmacologically acceptable” refer to molecular entities and compositions that do not produce an adverse, allergic or other untoward reaction when administered to an animal, or to a human, as appropriate. The term, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Pharmaceutically acceptable salts of the disclosed compounds or agents can be synthesized, for example, from a parent compound that contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704.

As used herein, the term “stereoisomers” refers to compounds made up of the same atoms bonded by the same bonds but having different spatial structures which are not interchangeable. The three-dimensional structures are called configurations. As used herein, the term “enantiomers” refers to two stereoisomers whose molecules are nonsuperimposable mirror images of one another. The terms “racemate,” “racemic mixture” or “racemic modification” refer to a mixture of equal parts of enantiomers.

### Methods and Compositions of the Invention

In general the invention provides methods for treating and/or controlling hepatitis C using one or more MTP inhibitors, for example, AEGR-733 or implitapide, in combination with at least one other active agent, e.g. a cholesterol, lipid, or lipoprotein lowering or inhibition agent, and/or a hepatitis C treatment. The MTP inhibitors can be used at dosages lower than those already found to result in one or more adverse events, for example, gastrointestinal disorders, abnormalities in liver functional and/or hepatic steatosis (for example, 25 mg/day of AEGR-733, 80 mg/day of implitapide and 160 mg/day of implitapide have been found to cause gastrointestinal disorders, abnormalities in liver function and/or hepatic steatosis) but still are therapeutically effective against hepatitis C, e.g. when combined with at least one other active agent. The dosages of the MTP inhibitors need not be smaller but may additionally and/or optionally be administered less frequently. Methods of treating hepatitis C using specific MTP inhibitors, e.g. implitipide, are also contemplated. It is contemplated that such MTP inhibitors, alone or in combination, may be effective at treating hepatitis C in a patient even when larger dosages of an MTP inhibitor is administered.

Methods of treating hepatitis C disclosed herein include methods of treating patients affected with particular genotypes of hepatitis C, e.g. genotype 1a, 1b, 2a, 2b, 3a, 3b, 4 and 5a, and/or includes methods of treating infections caused by antiviral resistant mutations of hepatitis C. Patients may include those patients previously treated for a hepatitis C infection, e.g. relapsed patients, and/or patients that did not respond to an initial treatment regimen that may or may not have included administering MTP inhibitors.

Contemplated herein are methods of treating acute and/or chronic hepatitis C. Acute hepatitis C typically refers to the first 6 months after infection with hepatitis C. Chronic hepatitis C typically refers to an hepatitis infection with hepatitis C that persists more than six months.

Also contemplated herein are methods of treating hepatitis C related disorders, such as those associated with, caused by, or result from, a chronic hepatitis C infection. Examples of such hepatitis C related disorders include B-cell non-Hodgkin's lymphoma, glomerulonephritis (e.g. membranoproliferative glomerulonephritis), cyroglobulimenia, thyroiditis, porphyria cutanea tarda, sicca syndrome, thrombocytopenia, lichen planus, arthritis, and cognitive disorders.

Also provided herein are compositions for use in the treatment of hepatitis C that include a MTP inhibitor and one or more other active agents, e.g. a cholesterol, lipid, or lipoprotein lowering or inhibition agent, and/or a hepatitis C treatment agent.

This invention also provides kits for conveniently and effectively implementing the methods of this invention. Such kits comprise a composition suitable for administration to a patient comprising an MTP inhibitor, and a means for facilitating compliance with methods of this invention. Kits may further comprise a hepatitis C treatment agent suitable for administration to a patient, e.g. comprising alpha-interferon and, together or separately, ribavirin. Such kits provide a convenient and effective means for assuring that the subject to be treated takes the appropriate active in the correct dosage in the correct manner. The compliance means of such kits includes any means which facilitates administering the actives according to a method of this invention. Such compliance means include instructions, packaging, and dispensing means, and combinations thereof. Kit components may be packaged for either manual or partially or wholly automated practice of the foregoing methods. In other embodiments involving kits, this invention contemplates a kit including compositions of the present invention, and optionally instructions for their use.

### Combination Therapies Using MTP Inhibitors

The method comprises a combination therapy, which can be achieved by co-administering to the mammal a MTP inhibitor and at least one other active agent. The MTP inhibitor and one or more other active agents can be administered as a (i) single dosage form or composition, (ii) simultaneously as separate dosage forms or pharmaceutical compositions, (iii) sequentially, as separate dosage forms starting with the MTP inhibitor and then administering the one or more other active agents, starting with the at least one other active agent and then administering the MTP inhibitor, or starting with at least one other active agent, then administering the MTP inhibitor, and then administering the same or different other active agent (iv) successively, separated by for example 1-4 hours, 1-8 hours or 1-12 hours, a day, or 2 or more days, e.g. 2 to 3 days, or (v) individually followed by the combination. The methods disclosed herein may occur before, during, or after other dosing regimens that may include, for example MTP inhibitors, a cholesterol, lipid, or lipoprotein lowering or inhibition agent, and/or a hepatitis C treatment. For example, a MTP inhibitor can be administered together with, e.g. a hepatitis C treatment agent for a period of time, e.g. 28 days, and the hepatitis C treatment can continue to be administered alone for a further administration time, e.g. 12 weeks.

Cholesterol, lipid or lipoprotein lowering or inhibition agents (referred to herein collectively as “cholesterol lowering agents”) include for example, HMG-CoA reductase inhibitors, bile acid sequestrants, fibric acid derivatives, niacin, squalene synthase inhibitors, ACAT inhibitors, and/or CETP inhibitors. Hepatitis C treatments or treatment agents include those therapies and agents developed for use in the treatment of hepatitis C, including, e.g. antiviral agents, inosine monophosphate inhibitors (e.g. ribavirin), alpha-interferon (IFNα) and pegylated alpha-interferon (PEG-IFN), including PEG-IFNα-2b and PEG-IFNα-2a. RNA polymerase inhibitors, and protease inhibitors. The term “at least one other active agent” includes, e.g. hepatitis C therapies or treatment agents that are themselves co-administered, e.g. PEG-IFN and ribavirin.

In some embodiments, the MTP inhibitor is administered in escalating doses. Such escalating doses may comprise a first dose level and a second dose level. In other embodiments, escalating doses may comprise at least a first dosage level, a second dosage level, and a third dosage level, and optionally a fourth, fifth, or sixth dosage level. The cholesterol absorption inhibitor may be provided in one dosage level when in administered in combination with a MTP inhibitor, or may be administered in escalating doses.

A first, second, third or more dosage levels can be administered to a patient for about 2 days to about 6 months or more in duration. For example, first, second and/or third dose levels are each administered to a subject for about 1 week to about 52 weeks, or about 1 week to about 24 weeks, or about 1 week to about twelve weeks. Alternatively, the first, second and/or third dosage levels are administered to a subject for about 2 days to about 60 days, to about 6 months, or to about 9 months.

In some embodiments, MTP inhibitors are administered as a short-term treatment, e.g. administered daily for 1 week, 5 weeks, nine weeks, while another active agent, e.g. PEG-IFN and ribavirin) is administered to a patient as a longer term treatment, e.g. 1 month, 4 months, 9 months.

The methods disclosed herein reduce the hepatitis C viral load of a patient. For example, after e.g. eight days, fourteen days, or 1 month of administering a disclosed therapy, a patient may have a 20%, 30%, 50%, 80%, 90% or more reduction in viral load, e.g. a patient may have an about 3 log₁₀ reduction in plasma hepatitis C virus RNA after two days and/or may have about 5.5 log₁₀ reduction in plasma hepatitis C virus RNA after 14 days of treatment. At an end point in treatment, patients may have viral levels below the limit of quantification (30 IU/ml) or even below the limit of detection (10 IU/ml).

The MTP inhibitor and/or the other active agent each may be administered in a therapeutically effective amount and/or each in a synergistically effective amount. Such dosages of a MTP inhibitor and/or the other active agent may, while not effective when used in monotherapy, may be effective when used in the combinations disclosed herein.

The precise time of administration and amount of any particular MTP inhibitor and/or any other active agent that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

Responses to treatment may be characterized by different viral kinetic profiles. Virological responders to treatment typically have a very rapid initial decrease in viral load, followed by a second, slower phase of decline until, e.g. undetectable levels of circulating virus are achieved. For hepatitis C, it is believed the initial decrease reflects the efficiency of suppression of replication and can be calculated as a percentage decline in hepatitis C RNA levels. The second phase response is believed to be due to clearance of virus-infected cells, by cell death or by eradication of viral replication in the cell, and can be calculated from the rate of decline in hepatitis C RNA levels following the first phase response. The MTP inhibitors and/or other active agents can be administered during a predicted first phase decrease in viral load, e.g. a MTP inhibitor can be administered during a predicted first phase decrease in viral load due to treatment by e.g. alpha-interferon, or a MTP inhibitor can be administered during a predicted slower second phase in viral load due to administrating alpha-interferon.

Treatment dosages or combination therapeutic protocols may be varied for different patient populations or viral type. Viral and patient related factors that affect response to treatment include baseline viral load, early virological response, presence of liver fibrosis/cirrhosis and HCV genotype.

Sustained virological response, or eradication of the infection is typically defined as continued undetectable serum HCV RNA levels six months after the completion of treatment. Sustained virological response can be used to compare different HCV therapies. In some embodiments, the methods disclosed herein result in fewer sustained virological responses in patients as compared to traditional therapies. One goal of HCV therapies is prevention of advanced liver diseases such as cirrhosis, hepatic failure, and hepatocellular carcinoma.

The methods disclosed herein can be tailored for patient infected with different different genotypes, e.g. infection with genotypes 2 or 3, or genotype 1. Alternatively, the methods can also be varied for patients with lower viral levels, or patients with acute vs. chronic infection.

Further, the disclosed methods can be varied based on host factors, e.g male or female patients, patient age, level of liver fibrosis, weight and/or body mass index, ethnicity, and/or presence or absences of other co-morbidities, e.g. alcohol abuse, renal disease or HIV infection.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations. Treatment may be initiated with smaller dosages which are less than the optimum dose of the compound. Thereafter, the dosage may be increased by small increments until the optimum therapeutic effect is attained.

### MTP Inhibitors

In one embodiment, the microsomal triglyceride transfer protein (“MTP”) inhibitor may be AEGR-733. As used herein, the phrase “BMS-201038” or “AEGR-733” refers to a compound known as N-(2,2,2-Trifluorethyl)-9-[4-[4-[[[4′-(trifluoromethyl) [1,1′biphenyl]-2-Yl]carbonyl]amino]-1-piperidinyl]butyl]9H-fluorene-9-carboxamide, having the formula:

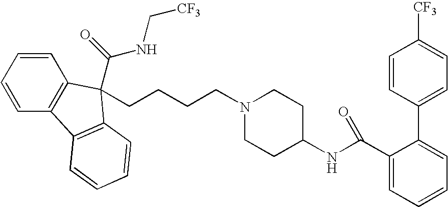

the piperidine N-oxide thereof, and stereoisomers, and pharmaceutically acceptable salts and esters thereof.

In another embodiment, the MTP inhibitor may include benzimidazole-based analogues of AEGR-733, for example, a compound having the formula shown below:

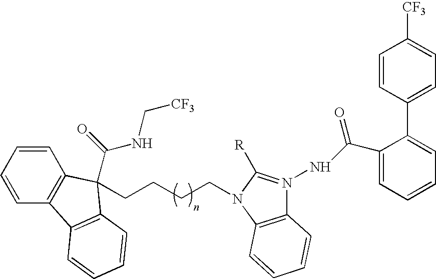

where n can be 0 to 10, and stereoisomers thereof, and pharmaceutically acceptable salts and esters thereof.

In another embodiment, the MTP inhibitor may be implitapide. As used herein, the phrase “implitapide” refers to a compound (2S)-2-cyclopentyl-2-[4-[(2,4-dimethyl-9H-pyrido[2,3-b]indol-9-yl)methyl]phenyl]-N-[(1S)-2-hydroxy-1-phenylethyl]ethanamide, and having the structure shown below:

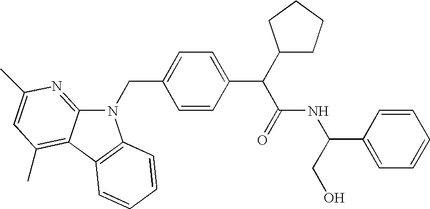

and stereoisomers thereof; and pharmaceutically acceptable salts and esters thereof.

In another embodiment, the MTP inhibitor may be JTT-130m including pharmaceutically acceptable salts and esters thereof, described in Aggarwal, et al., BMC CARDIOVASC. DISORD. 27; 5(1):30 (2005). In another embodiment, the MTP inhibitor may be CP-346086 including pharmaceutically salts and esters thereof, described in Chandler, et al., J. LIPID. RES. 44(10):1887-901 (2003). Other MTP inhibitors include those developed by Surface Logix, Inc. e.g., SLx-4090.

### Other Active Agents

A. Cholesterol Lowering Agents

Other active agents include cholesterol lowering agents such as cholesterol absorption inhibitors (CAI). In one embodiment, the CAI may be ezetimibe (also known as Zetia). As used herein, the phrase “ezetimibe” refers to a compound having the structure shown below:

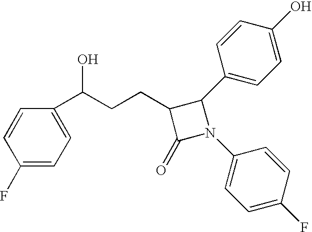

and stereoisomers thereof, and pharmaceutically acceptable salts and esters thereof. Ezetimibe can be co-administered with a MTP inhibitor at a dosage in the range of 0.01 to 100 mg/day, more preferably at a dosage in the range of 1 to 50 mg/day. For example, ezetimibe may be administered at a dosage of 10 mg/day.

In one embodiment, the CAI may be MD-0727 including pharmaceutically acceptable salts and esters thereof. In another embodiment, the CAI may be FM-VP4. As used herein, the phrase “FM-VP4” refers to a compound the structure of which is set forth below:

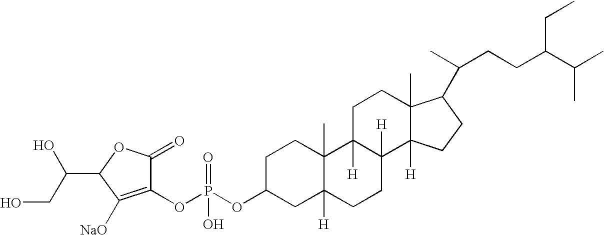

and stereoisomers thereof, and pharmaceutically acceptable salts and esters thereof.

Alternatively, the CAI may be represented by the structure below, as described in Ritter et al., Org. Biomol. Chem., 3(19), 3514-3523, (2005):

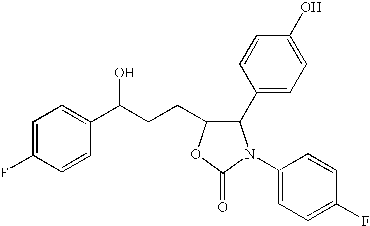

and stereoisomers thereof, and pharmaceutically acceptable salts and esters thereof.

In another embodiment, the CAI may be LPD 179. As used herein, the phrase “LDP179” refers to a compound having the structure set forth below:

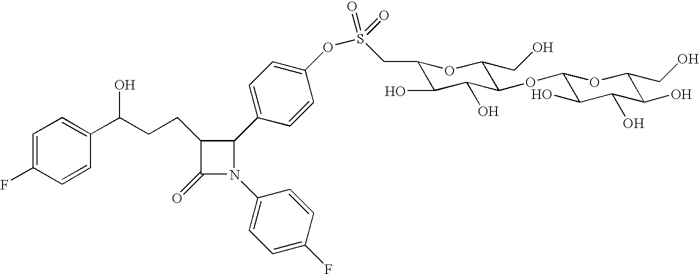

and stereoisomers thereof, and pharmaceutically acceptable salts and esters thereof.

In another embodiment, the CAI may be LPD84. As used herein, the phrase “LPD84” refers to a compound having the structure set forth below:

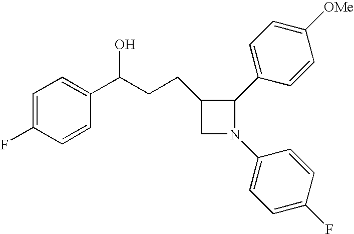

and stereoisomers thereof, and pharmaceutically acceptable salts and esters thereof.

In another embodiment, the CAI may be LPD145. As used herein, the phrase “LPD145” refers to a compound having the structure set forth below:

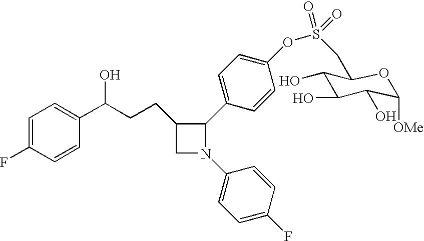

and stereoisomers thereof, and pharmaceutically acceptable salts and esters thereof.

Other useful exemplary ezetimibe derivatives, their synthesis and use are described, for example, in International Application Publication No. WO 2005/033100.

The pharmaceutical compositions and methods disclosed herein can further comprise one or more HMG CoA reductase inhibitors, also known as “statins.” Statins slow down the body's production of cholesterol and inhibit the bioconversion of hydroxymethylglutaryl-coenzyme A to mevalonic acid catalyzed by the enzyme HMG-CoA reductase. A HMG-CoA reductase inhibitor can be administered at a dosage of 0.01 to 100 mg/day, optionally, 1 to 50 mg/day, optionally 1 to 25 mg/day. For example, a HMG-CoA reductase inhibitor is administered at a dosage of 5 mg/day, 10 mg/day, 20 mg/day, 40 mg/day, or 85 mg/day.

In one embodiment, the statin can include atorvastatin. As used herein, the term “atorvastatin” refers to a compound known in the art as atorvastatin (7-[2-(4-fluorophenyl)-5-(1-methylethyl)-3-phenyl-4-(phenylcarbamoyl)-1H-pyrrol-1-yl]-3,5-dihydroxy-heptanoic acid; brand name: Lipitor) and pharmaceutically acceptable salts and esters thereof. Atorvastatin can be administered at, e.g. 10 mg/day.

Fluvastatin is also contemplated. As used herein, the term “fluvastatin” refers to a compound known in the art as fluvastatin (sodium 7-[3-(4-fluorophenyl)-1-propan-2-yl-indol-2-yl]-3,5-dihydroxy-hept-6-enoate; brand name: Lescol) and pharmaceutically acceptable salts and esters thereof.

In another embodiment, the statin may be lovastatin. As used herein, the term “lovastatin” refers to a compound known in the art as lovastatin (8-[2-(4-hydroxy-6-oxo-tetrahydropyran-2-yl)ethyl]-3,7-dimethyl-1,2,3,7,8,8a-hexahydronaphthalen-1-yl]2-methylbutanoate; brand names: Altocor, Mevacor) and pharmaceutically acceptable salts and esters thereof.

In another embodiment, the statin may be pravastatin. As used herein, the term “pravastatin” refers to a compound known in the art as pravastatin (5-dihydroxy-7-[6-hydroxy-2-methyl-8-(2-methylbutanoyloxy)-1,2,6,7,8,8a-hexahydronaphthalen-1-yl]-heptanoicacid; brand name: Pravachol) and pharmaceutically acceptable salts and esters thereof.

In another embodiment, the statin may be rosuvastatin. As used herein, the term “rosuvastatin” refers to a compound known in the art as rosuvastatin (7-[4-(4-fluorophenyl)-6-(1-methylethyl)-2-(methyl-methylsulfonylamino)-pyrimidin-5-yl]-3,5-dihydroxy-hept-6-enoic acid; brand name: Crestor) and pharmaceutically acceptable salts and esters thereof. Statins also include pitavastatin, tenivastatin, rivastatin, mevastatin, and cerivastatin, and pharmaceutically acceptable salts and esters thereof.

In another embodiment, the statin may be simvastatin. As used herein, the term “simvastatin” refers to a compound known in the art as simvastatin (7-(2,6-dimethyl-8-(2,2-dimethylbutyryloxy)-1,2,6,7,8,8a-hexahydro-1-naphthyl)-3,5-dihydroxyheptanoic acid; brand name: Zocor) and pharmaceutically acceptable salts and esters thereof.

Other active agents, e.g. cholesterol lowering agents, include bile acid sequestrants, also known as resins. Bile acid sequestrants help lower levels of LDL. In one embodiment, the bile acid sequestrants may be cholestyramine (brand names: Locholest, Prevalite, Questran), colesevelam (brand name: Weichol), or colestipol (brand name: Colestid) including pharmaceutically acceptable salts and esters thereof.

Fibrates (also known as fibric acid derivatives) help lower the cholesterol by reducing the amount of triglycerides (fats) in the body and by increasing the level of “good” cholesterol (also called HDL, or high-density lipoprotein). In one embodiment, the fibrate may be fenofibrate (1-methylethyl2-[4-(4-chlorobenzoyl)-phenoxy]-2-methyl-propanoate; brand name: Tricor), bezafibrate, ciprofibrate, clofibrate, or gemfibrozil (5-(2,5-dimethylphenoxy)-2,2-dimethyl-pentanoicacid; brand name: Lopid), including pharmaceutically acceptable salts and esters thereof. It is contemplated that the methods may include administration of niacin (also called nicotinic acid), which is a B vitamin.

Squalene synthase inhibitors include compounds which inhibit the condensation of molecules of farnesylpyrophosphate to form squalene, catalyzed by the enzyme squalene synthase. Inhibition is readily determined by those skilled in the art according to standard assays (e.g., Meth. Enzymol, 15, 393-454 (1969) and Meth. Enzymol, 110, 359-373 (1985)). A variety of these compounds are known to those skilled in the art, e.g., in U.S. Pat. No. 5,026,554, disclosing fermentation products of the microorganism MF5465 (ATCC 74011) including zaragozic acid. A summary of other squalene synthase inhibitors has been compiled (Curr. Op. Ther. Patents, 3, 861-4 (1993)). In one embodiment, the squalene synthase inhibitor may be TAK-475, ER-27856, or RPR-107393 including pharmaceutically acceptable salts and esters thereof.

ACAT inhibitors refer to compounds that inhibit the intracellular esterification of dietary cholesterol by the enzyme acyl CoA: cholesterol acyltransferase. Such inhibition may be determined readily by one of skill in the art according to standard assays, such as the method described in Heider et al., Journal of Lipid Research., 24, 1127 (1983). A variety of these compounds are well known to those skilled in the art, e.g., U.S. Pat. No. 5,510,379 (carboxysulfonates), WO 96/26948 and WO 96/10559 (urea derivatives having ACAT inhibitory activity); DL-melinamide (GB Pat. No. 1,123,004 and Japan. J. Pharmacol., 42, 517-523 (1986); 2,2-dimethyl-N-(2,4,6 trimethoxyphenyl)dodecanamide (U.S. Pat. No. 4,716,175); and N-[2,6-bis(1methylethyl)phenyl]-N′-[[1-(4-dimethylaminophenyl)cyclopentyl]-methyl urea (U.S. Pat. No. 5,015,644). In one embodiment, the ACAT inhibitor may be avasimibe, pactimibe, or HL-004, including pharmaceutically acceptable salts and esters thereof.

CETP inhibitors include compounds that inhibit the cholesterol ester transfer protein (CETP)-mediated transport of various cholesteryl esters and triglycerides from HDL to LDL and VLDL. Such CETP inhibition activity is readily determined by those skilled in the art according to standard assays (e.g., U.S. Pat. No. 6,140,343). A variety of CETP inhibitors will be known to those skilled in the art, including U.S. Pat. Nos. 6,140,343 (4-amino substituted-2-substituted-1,2,3,4 tetrahydroquinolines); 5,512,548 (polypeptide derivatives) and CETP-inhibitory rosenonolactone derivatives and phosphate-containing analogs of cholesteryl ester (J. Antibiot., 49(8), 815-816 (1996), and Bioorg. Med. Chem Lett., 6, 1951-1954 (1996), respectively.) In one embodiment, the CETP inhibitor may be torcetrapib or JTT-705.

Other classes of compounds that can be used in combination with a MTP inhibitor includes PPAR (peroxisome proliferator activated receptor) alpha, delta, or gamma agonists such as muraglitazar, anti-inflammatory agents; LXR (liver X receptor), FXR, and RXR (retinoid X receptor) agonists; ABC (ATP binding cassette) transporters; and CB-1 (cannaboid)antagonists such as rimonabant (5-(4-Chlorophenyl)-1-(2,4-dichlorophenyl)-4-methyl-N-(piperidin-1-yl)-1H-pyrazole-3-carboxamide; Sanofi-Synthelab). Also contemplated compounds for use in the compositions and methods of this disclosure include ω-3 fatty acids, ileal bile acid co-transporters and inhibitors of same (IBATs), niacin receptor agonists, metformin, DPP-IV antagonists, sulphonylurea (SU), FAB protein inhibitors, and GLP-1 agonists.

B. Hepatitis C Treatment Agents

Hepatitis C treatment agents include alpha-interferon-2b (brand name: Intron-A) and alpha interferon-2a (brand name: Roeferon-A) which are recombinant alpha interferons. Monotherapy with such interferons is typically a 24-48 week or more course of 3 million international units administered subcutaneously three times weekly. Interferon for use as a hepatitis treatment agent can include pegylated interferon, which can be administered with a once weekly injection up to 48 weeks. Albuferon is a long acting form of alpha-interferon, which may be administered as 900 mcg or 1200 mcg biweekly or every four weeks.

When, e.g. combined with interferon or pegylated interferon, ribavirin synergistically lowers the level of hepatitis C RNA viral levels, and can be used together as hepatitis C treatment agent. For example, 3 million international units of alpha-interferon can be administered three times weekly together with ribavirin, e.g. 800-2000 mg daily, for 48 weeks. For certain hepatitis C genotypes, e.g. genotypes 2 and 3, for example, pegylated interferon can be administered three times weekly together with e.g. 800 mg daily of ribavirin for 24 weeks.

Ribovarin can be classified as an inosine monophosphate dehydrogenase inhibitor.

Other inosine monophosphate dehydrogenase inhibitors contemplated as active agents, alone or in combination with administration of an alpha-interferon, include VX-497 (Vertex Pharmaceuticals), viramidine (pro-drug for ribavirin), mycophenolate mofetil, tiazofurin, mizoribine, and taribavirin. Other agents that may be used in combination with MTP inhibitors include hepatitis C virus protease inhibitors, e.g. NS3/4A serine protease inhibitor. The NS3-4A protease activity is believed to block a host cell's ability to mount an innate antiviral response. Inhibitors of this enzyme may block virus replication, leading to increased antiviral efficacy. NS3 or 4A protease inhibitors include VX-950 (Vertex. Pharmaceuticals), SCH-7 (SCH 503034) (Schering), GS-9132/ACH-806 (Gilead), ITMN-191 (Roche), and BILN-2061. For example, VX-950 may be administered as 450 mg or 750 mg every 8 hours for e.g 14 days, 28 days, or more.

Hepatitis polymerase inhibitors are also contemplated as other active agents. The RNA-dependent RNA polymerase (RdRp) contained within the NS5B protein is the catalytic component of the hepatitis replication machinery and thus plays an essential role in viral replication. Nucleoside analogs, such as 2′-C-methyl purine nucleosides can inhibit hepatitis C, as well as non-nucleoside inhibitors. Non-nucleoside NS5B RNA polymerase inhibitors include benzothiadiazines, benzimidazoles, JTK-002, JTK-109, JTK-003 (Japan Tobacco), HCV-796 (Wyeth), valopicitabine (Novartis), R1626 (Roche), XTL-2125 (XTL Biopharmaceuticals), and VCH-759.

Small interfering RNA may be a hepatitis C agent. Hepatitis is a positive-strand RNA virus that replicates via a double-stranded RNA intermediate. Hepatitis C virus antigen expression is specifically silence by siRNAs targeting the hepatitis C virus positive strand. siRNA agents include SIRNA-034 (Sirna Therapeutics), and TT-0033i (Tacere Therapeutics).

MicroRNA known as miR-122 appears to be required for hepatitis C virus replication in mammalian cells. microRNA (miR-122) inhibitors are therefore contemplated as active agents for use in the disclosed methods.

Toll-like receptor inhibitors are also contemplated, such as ANA975 (Anadys), isatoribine, and Actilon (Coley Pharmaceuticals).

Other contemplated agents include celgosivir, castanospermine, thymalfasin, bavituximab, NIM811, nitazoxanide, GSK625433 (Glaxo SmithKline), alpha-glucosidase inhibitors, e.g. celgosivir, ribosome entry site inhibitors, e.g. mifepristone, immunomodulators, e.g. NOV-205 (Novalose), AVI-4065, NM-283, amantadine, and pioglitazone.

Hepatitis C treatment agents include vaccines. Vaccine products may require multiple components that target various aspects of immunity. Such vaccines include Civacir (Nabi Pharmaceuticals) HEPAVAXX C (ViRexx), IC41 (Intercell AG), PEV2A, PEV2B, GI-5005, and HCV E1 (Innogenetics).

### Therapies Using AEGR-733

In one aspect, the invention provides a method of treating and/or controlling hepatitis C comprising administering a combination therapy that includes administering AEGR-733 to a patient daily, and administering at least one other active agent.

Exemplary dosages for administration of AEGR-733, for example, in combination with a another active agent, e.g. ezetimibe, VX-950, and/or pegylated interferon include a dosage of about 1 mg/day to about 25 mg/day, e.g. 2.5 mg/day, 5 mg/day, 7.5 mg/day, 10 mg/day, 15 mg/day or 20 mg/day of AEGR-733. Additional active agents may also be administered to patient, e.g. a MTP inhibitor and ezetimibe may each be administered daily to a patient for e.g. 1 week, 6 weeks, or even 9 weeks, and a course of interferon and optionally ribavirin may also be administered to a patient before, during, or after administration of, e.g. AEGR-733 and ezetimibe.

In an exemplary dose escalation regimen, the first dose level of AEGR-733 may be from about 2 to about 13 mg/day, and/or the second dose level may be about 5 to about 30 mg/day.

In an exemplary protocol, AEGR-733 initially is administered at a first dosage in the range of 2.5 to 7.5 mg/day for at least 4 weeks, is then administered at a second dosage in the range of 5 to 10 mg/day for at least 4 weeks, and is then administered at a third dosage in the range of 7.5 to 12.5 mg/day for at least 4 weeks. Such dosage regimens may be in combination with 10 mg/day of ezetimibe and/or about 750 mg/TID of VX-950.

The first dosage of AEGR-733 can be for example 2.5 mg/day or 5 mg/day. The second dosage of AEGR-733 can be 7.5 mg/day. The third dosage of AEGR-733 can be 10 mg/day. In certain embodiments, the second dosage is administered immediately following the first dosage, i.e., the second dosage is administered starting at five weeks from the initial first dosage. Similarly, in certain other embodiments, the third dosage of AEGR-733 is administered immediately following the second dosage, e.g., the second dosage is administered at nine weeks from the initial first dosage.

Optionally, the method may include administering a fourth dosage of AEGR-733 alone, or in combination with ezetimibe. Such a fourth dosage may be in the range of 7.5-12.5 mg/day or more. A fourth dosage may occur immediately after the third dosage, or may occur after a time interval, for example, a day, days, a week, or weeks after the third dosage. The fourth dosage may be administered to the subject for 1, 2, 3, 4 or more weeks.

For example, a patient can be treated with about 2.5 mg/day to about 12.5 mg/day of AEGR-733 in combination with administration of pegylated interferon (and optionally ribavirin) for about 14 days or about 28 days, and optionally then treated or continued with pegylated interferon alone for, e.g. about 12 weeks or more.

### Therapies Using Implitapide

In one aspect, the invention provides a method of treating and/or controlling hepatitis C comprising administering implitapide to a patient daily, and optionally administering at least one other active agent.

Implitapide may be administered at a dosage in the range of 0.01 to 60 mg/day, more preferably in the range of 20 to 60 mg/day, for example, 20 mg/day, 25 mg/day, 30 mg/day, 35 mg/day, 40 mg/day or 60 mg/day.

For example, a patient can be treated with about 20 mg/day to about 60 mg/day of implitapide in combination with administration of pegylated interferon (and optionally ribavirin) for about 14 days or about 28 days, and optionally then treated or continued with pegylated interferon and/or ribavirin alone for, e.g. about 12 weeks or more.

### Formulation and Administration of the Active Ingredients

In certain embodiments, the MTP inhibitor (for example, AEGR-733 and implitapide) and the other active agent (for example, ezetimibe and/or VX-950) are administrated orally. For oral administration, the active ingredients may take the form of solid dose forms, for example, tablets (both swallowable and chewable forms), capsules or gelcaps, prepared by conventional means with pharmaceutically acceptable excipients and carriers such as binding agents (e.g. pregelatinised maize starch, polyvinylpyrrolidone, hydroxypropylmethylcellulose and the like), fillers (e.g. lactose, microcrystalline cellulose, calcium phosphate and the like), lubricants (e.g. magnesium stearate, talc, silica and the like), disintegrating agents (e.g. potato starch, sodium starch glycollate and the like), wetting agents (e.g. sodium laurylsulphate) and the like. Such tablets may also be coated by methods well known in the art.

In other embodiments, it is contemplated that the MTP inhibitor is administered orally, while the other active ingredients may be formulated for, and administered by, non-parental routes, for example, by intravenous routes, intramuscular routes, and by absorption through mucous membranes. It is contemplated that such formulations and non-parenteral modes of administration are known in the art.

An MTP inhibitor may be administered before, substantially simultaneously, or after administration of a vaccine.

In certain embodiments, the methods disclosed herein may minimize at least one of side effects associated with the administration of AEGR-733 and/or implitapide. Such side effects include, for example, diarrhea, nausea, gastrointestinal disorders, steatorrhea, abdominal cramping, distention, elevated liver function tests such as increases in liver enzymes such as alanine, minor fatty liver; hepatic fat build up, polyneuropathy, peripheral neuropathy, rhabdomyolysis, arthralgia, myalgia, chest pain, rhinitis, dizziness, arthritis, peripheral edema, gastroenteritis, liver function tests abnormal, colitis, rectal hemorrhage, esophagitis, eructation, stomatitis, biliary pain, cheilitis, duodenal ulcer, dysphagia, enteritis, melena, gum hemorrhage, stomach ulcer, tenesmus, ulcerative stomatitis, hepatitis, pancreatitis, cholestatic jaundice, paresthesia, amnesia, libido decreased, emotional lability, incoordination, torticollis, facial paralysis, hyperkinesia, depression, hypesthesia, hypertonia, leg cramps, bursitis, tenosynovitis, myasthenia, tendinous contracture, myositis, hyperglycemia, creatine phosphokinase increased, gout, weight gain, hypoglycemia, anaphylaxis, angioneurotic edema, and bullous rashes (including erythema multiforme, Stevens-Johnson syndrome, and toxic epidermal necrolysis). In some embodiments the minimization of the side effect is determined by assessing the grade, severity, extent, or duration by subject questionnaire.

### Assays

MTP inhibitors, optionally in combination with other active agents, that block or reduce HCV particle release may be identified using cell-based assays. Cells expressing HCV RNA, such as HCV replicons are treated with a MTP inhibitor at a range of doses. Exemplary cells may include Huh7 cells, Huh7.5 cells (a mutant line of Huh7 cells capable of supporting HCV replication at high efficiency), Huh7-K2040 cells, Huh7-5A-GFP-6 cells (a transformed cell line expressing genotype 1b HCV subgenomic replicons), and Huh7-GL cells (a transformed line of Huh7 cells containing a chromosomally integrated genotype 2a HCV cDNA that constitutively produces infectious virus). Huh7 and Huh7.5 cells may be maintained in medium A, which contains DMEM with 4.5 g/liter glucose, 100 units/ml penicillin, 100 μg/ml streptomycin sulfate, and 10% fetal calf serum (FCS). Huh7-K2040 cells and Huh7-5A-GFP-6 cells are maintained in medium A supplemented with 200 μg/ml G418. Huh7-GL cells may be maintained in medium A supplemented with 5 μg/ml blasticidine. Cells are maintained in monolayer culture at 37° C. in 5% CO₂.

Huh7-GL cells can be incubated in the presence or absence of a MTP inhibitor. Varying concentrations of MTP inhibitors and/or other active agents may be tested in parallel and may range from 0.01 nM to 100 nM. Incubation times may also be tested in parallel and may range from less than 1 hour to 72 hours, such as 0 hour, 0.5 hour, 1 hour, 4 hours, 16 hours, 24 hours, 36 hours, 48 hours, and 72 hours.

Other assays for identification of MTP inhibitors alone or in combination block or reduce HCV include hepatitis C virus replicons which incorporate a reporter such as luciferase. Other assays that can be used alone or in combination with other assays include luciferase assays, green fluorescent protein assays, cytotoxicity assays, and cholesterol biosynthesis assays.

### EXAMPLES

The examples that follow are intended in no way to limit the scope of this invention but are provided to illustrate the methods present invention. Many other embodiments of this invention will be apparent to one skilled in the art.

### Example 1

### Assay for Identifying MTP Inhibitor Combinations for Blocking HCV Particle Release

On day 0, huh7-GL cells are set up at 7×10⁵ cells per 60-mm dish. On day 1, cells are treated with or without the MTP inhibitor and/or other active agents. Approximately twelve to sixteen hours later on day 2, cells are switched to serum-free medium in the presence and absence of the MTP inhibitor. The concentration of MTP inhibitor and/or other active agents is the same in serum-free medium as used in serum-containing medium. Cells are then incubated with for a set time, such as 4 hours.

Following incubation, the culture medium is harvested and subjected to SDS-PAGE followed by an immunoblot using protocols well-known in the art. Antibodies that may be used to detect proteins of interest may include, for example, anti-apoB, anti-apoE, and anti-MTP. Control antibodies, such as anti-α1-antitrypsin, may also be used. A sheep polyclonal anti-apoB antibody and an anti-α1-antitrypsin antibody is available Biodesign International (Kennebunkport, Me.). A goat polyclonal anti apoE antibody is available from CalBioChem (San Diego, Calif.).

Following incubation, the culture medium may also be harvested and the amount of HCV RNA may be measured by real-time PCR. To measure intracellular RNA, HCV RNA is extracted from cells following standard protocols and first strand cDNA is synthesized from the extracted RNA and subjected to real-time PCR quantification using HCV RNA specific primers. Samples of cDNA are amplified in triplicate. Human 36B4 may be used as an invariant control and the relative amounts of RNAs are calculated using a comparative C_{T} method.

To measure extracellular RNA, RNA is extracted from the culture medium with a QIAamp Viral RNA Mini Kit (Qiagen). First strand cDNA is then synthesized using extracted RNA or in vitro transcribed HCV RNA. Samples of cDNA, in triplicate, are then subjected to real-time PCR quantification using HCV RNA specific primers. HCV copy numbers are determined by a standard curve generated using the in vitro transcribed HCV RNA. The conversion constant is that 1 μg of single-stranded RNA equals 1.96×10¹¹ viral copy number (Ambion Technical Bulletin 165 available on the world wide web with the extension ambion.com/techlib/tb/tb_{—}165.html).

Also following incubation, HCV titer may be determined by measuring foci formation. Harvested cells are plated and incubated in medium A for approximately 16 to 24 hours. For example, 1.5×10⁴ cells may be plated per well in a 48-well plate. Medium A is then removed and replaced with HCV-containing medium A without serum supplementation. For determining titer, it is convenient to add 100 μl of HCV-containing medium A without serum per well. Cells are then incubated for 2 hours at 37° C. Following incubation, cells are washed in PBS, fixed with PBS containing 4% paraformaldehyde for 30 minutes, washed in PBS containing 10 mM glycine, permeabilized with PBS containing 0.2% Triton X-100 for 15 minutes, and then incubated with PBS containing 10% FCS for 10 minutes. Cells are then incubated with human anti-HCV serum followed by a secondary antibody, such as HRP conjugated donkey anti-human IgG. Cells may be visualized using methods well-known in the art and stained cells are counted in each well. The number of stained cells is the foci formation unit (ffu) per 100 μl of culture medium. HCV titer is expressed as the number of ffu/ml.

### Example 2

### In Vitro Model of Hepatitis C Replication

The hepatitis C virus replicon (Huh 5-2 [I₃₈₉luc-ubi-neo-NS3-3′/5.1]) is an in vitro model of HCV replication in which the luciferase reporter is incorporated into HCV sequences (Lohmann et al, Science (1999) 285, 110-113; Krieger et al, J. Virol. (2001) 75, 4614-4624). The firefly luciferase reporter is expressed as a luciferase-ubiquitin-neomycin phosphotransferase fusion protein, which is cleaved by host proteases to release luciferase. The replicon also contains an internal EMCV IRES for translation of HCV NS3-5B polyprotein, which harbours cell culture adapted mutations to permit high cloning efficiency. The luciferase output is directly proportional to the level of HCV replicon RNA genomes present in the host cell, which can be directly measured by quantitative RT-PCR using a Taqman assay. Taqman probes and primers can be designed using Primer Express software (PE Biosystems) as outlined, for example, in appendix C of Taqman Universal PCR Master Mix protocol p/n 43044449 Rev B. This hepatitis C virus replicon system can been used to assay the antiviral effect of MTP inhibitors alone and in combination with other agents. Quantitative IC₅₀ data for both efficacy and toxicity can be obtained.

### Example 3

Replicon cells are passaged to maintain cells at 50-90% subconfluence. Cells are then trypsinsed and resuspended at 5.55×10⁴ cells/ml in DMEM complete. Aliquots (180 μL) containing 10⁴ cells are added to a clear 96 well plate (e.g for WST cytotoxic assay and RNA extraction) and a duplicate white/clear Wallac Isoplate (e.g for luciferase assay). An additional clear 96 well plate is set up for BrdU uptake. The plates ware incubated at 37° C., 5% CO₂ for 18 hours. A 10× dilution series can be generated in complete DMEM/10% DMSO in a 96 well plate in 9 three-fold steps from a 50 mM stock concentration. MTP inhibitors and/or other active agents (20 μL) are added to triplicate wells containing overnight seeded lucubineo cells and incubated for a further 72 hours at 37° C., 5% CO₂.

### Example 4

A luciferase assay can be performed. The cells are washed with 200 μL, PBS and all traces of PBS are removed before adding 25 μL Passive Lysis Buffer (Promega Lucifase assay kit E1501 as described in Tech bulletin 281). The cells are lysed for 30 minutes at room temperature before addition of 100 μL assay reagent. Light output is measured on a Victor luminometer and data stored for analysis.

### Example 5

A green fluorescent protein assay can be performed. Replicon cells are electroporated in cytomix buffer at 10₇ cells/mL at 960 uF, 270 V with varying amounts pIRES2-EGFP. Cell viability is monitored by trypan blue staining and cell counting. Aliquots of cells (2×10⁴) are plated out in clear bottomed black 96 well plates and incubated for 24 hours. The cells are rinsed with PBS and fluorescence output read using, e.g. an Analyst HT (LJL Biosystems).

### Example 6

Cytotoxity Assay and Quantitative RT-PCR can be performed. Statin-treated cells in the duplicate clear plate are analysed for cytotoxicity by addition of 10 μL WST-1 reagent (Roche Biosciences) to each well and incubated for 37° C. for 60 minutes. After vigorous shaking, the plate is read at 450 nm. The supernatent is removed and the monolayer washed with PBS prior to addition 100 μL RLT lysis buffer (Qiagen RNAeasy kit) to each test well, followed by vigorous resuspension to ensure uniform lysis of all cells. Total RNA is extracted according to Qiagen RNAeasy protocols recommended by manufacturer. Briefly, 0.1 μg total RNA is reverse transcribed using random hexamers and 20% cDNA that can be produced is used as a template in a Taqman reaction (e.g. Universal PCR Master Mix protocol p.backslash.n 43044449 Rev B).

A cytostaticity assay (BrdU Uptake Assay) can be performed. The Biotrak RPN 250 assay kit is used to perform this assay. 20 μL BrdU stock solution (100 .mu.M,) is added to the wells of the third 96 well plate and cells re cultured for 2 hours. The supernatent is removed and 200 μL fixative is added prior to incubation with peroxidase labelled anti-BrdU and further developed according to manufacturers instructions.

A cholesterol biosynthesis assay can be performed. Replicon cells are plated at a density of 2×10₅ per well in 450 μL complete medium in 24 well plates. The wells are treated with MPT inhibitors and/or other active agents. After 72 hours in culture the 24 well plates are taken out of the CO₂ incubator and 20 μL ^{14C}acetate (e.g. activity mCi/5 mL) is added to each well. The plates are sealed with parafilm to avoid evaporation and incubated at 37° C. for 6 hrs on an orbital shaker at 25 rpm. The samples are saponified; by adding 1 mL 5M KOH (in 100% MeOH) to each well, resealing, incubating at 70° C. for 2 hrs and then overnight at room temperature. After the saponification is completed the media plus cells from the wells is transferred to a 15 mL glass round bottom tube and extracted with 4.0 mL hexane by vortexing for 10 sec. and separating the organic phase (top) into a clean glass tube. This extraction can be repeated once more. Both organic phases of the same sample are pooled and dried under nitrogen. 1 mL hexane is added to the dry lipids vortexed well for 10 seconds and dried under nitrogen.

Following extraction, the dry samples are resuspended in chloroform (30 mL) by vigorous vortexing for 10 seconds and spotted onto 60A silica gel TLC plates. ^{14C}cholesterol (0.1 μCi) is spotted onto an empty lane as a marker. The plates are developed in a solvent system of hexane:diethylether:acetic acid (70:30:2). The plates are developed until the solvent front is within 0.5 cm of the top of the TLC plate (approx 1 hr). After the chromatography is completed the plates are air dried in a fume hood for 1 hour at room temperature.

The TLC plates can then be exposed to a phospho-screen for 24 hr. A Molecular Dynamics ‘STORM’ analyzer can be used to scan the screens. The scanned image is then visually inspected and the cholesterol bands located according to the position of the ^{14C}cholesterol marker.

### Example 7

### AEGR-733 Combination Therapy

This study is designed to show that doses of AEGR-733 in combination with other agents, e.g. ezetimibe and/or alpha-interferon, can provide clinically significant effects for the treatment of hepatitis C. The primary parameter of efficacy in this study is the reduction in viral load after 12 weeks of therapy.

Approximately 20 treatment-naïve subjects with stable levels of hepatitis C infection, i.e. viral load, and who have no evidence of liver disease are randomized into one of four treatment arms with equal probability. The subjects may have a median serum viral load at study entry of between 6.13 log₁₀ and 6.48 logᵢₙ hepatitis C virus RNA (˜1.5-3 million IU/mL). In treatment arm 1, subjects receive AEGR-733 (5 mg) plus standard alpha-interferon and ribavirin therapy. Treatment arm 1 patients, in effect, receive a MTP combination therapy. In treatment arm 2, subjects receive an AEGR-733 placebo plus standard alpha-interferon and ribavirin therapy. In effect treatment arm 2 represents monotherapy with alpha-interferon. In treatment arm 3, subjects receive an AEGR-733 (5 mg) capsule. In effect, treatment arm 3 represents monotherapy with AEGR-733. In treatment arm 4, patients receive a AEGR-733 placebo, and no other hepatitis therapy.

After 28 days of treatment, subjects are assessed for viral loads. Thereafter, subjects in arms 1 discontinue use of AEGR-733 and continue with use of standard alpha-interferon and ribavirin therapy for a total of 28 weeks of therapy. Throughout the study, viral amounts of hepatitis C of the subjects are measured as part of vital signs collection.

### EQUIVALENTS

It is understood that the disclosed invention is not limited to the particular methodology, protocols, and dosages described as these may vary. It is also to be understood that the terminology used herein is for the purpose of describing particular embodiments only, and is not intended to limit the scope of the present invention which will be limited only by the appended claims.

### INCORPORATION BY REFERENCE

The entire disclosure of each of the patent documents and scientific articles referred to herein is incorporated by reference for all purposes.

### Patents/Patent Applications

WO 2005/087234, U.S. Ser. No. 11/582,835, PCT/US06/40953

### PUBLICATIONS

Huang et al., PNAS v. 104 5848-5853 (2007).

What is claimed is:

1. A method of treating a hepatitis C infection, comprising administering to a patient in need thereof: i) a MTP inhibitor and ii) at least one other active agent.

2. The method of claim 1, wherein the at least one other active agent is a hepatitis C treatment agent and/or a cholesterol lowering agent.

3. The method of claim 1, wherein the at least one other active agent is selected from at least one of: a cholesterol absorption inhibitor, a HMG-CoA reductase inhibitor, a bile acid sequestrant, a fibric acid derivative, niacin, a squalene synthase inhibitor, an ACAT inhibitor, a CETP inhibitor, an inosine monophosphate dehydrogenase inhibitor, a NS3 or 4A protease inhibitor, alpha-interferon, pegylated alpha-inteferon, a NS5B RNA polymerase inhibitor A-689, A-831, siRNA, a miR-122 inhibitor, a toll-like receptor inhibitor, celgosivir, thymalfasin, bavituximab, NIM811, nitazoxanide, GSK625433, alpha-glucosidase inhibitors, ribosome entry site inhibitors, immunomodulators, AVI-4065, NM-283, amantadine, pioglitazone and a hepatitis C vaccine.

4. The method of claim 1, wherein the patient is a human.

5. The method of claim 1, wherein the hepatitis C infection is a chronic hepatitis C infection.

6. The method of claim 1, wherein the MTP inhibitor and the other active agent is administered sequentially.

7. The method of claim 1, wherein the MTP inhibitor and the other active agent is administered substantially simultaneously.

8-10. (canceled)

11. The method of claim 1, wherein administering the MTP inhibitor in combination with the other active agent provides a synergistic therapeutic effect.

12. The method of claim 11, wherein the MTP inhibitor is administered in a synergistically effective amount.

13. The method of claim 11, wherein the other active agent is administered in a synergistically effective amount.

14. The method of claim 1, wherein at least two other active agents are administered.

15. The method of claim 1, wherein at least two of the active agents are administered substantially simultaneously.

16. The method claim 1, wherein the MTP inhibitor is administered substantially simultaneously with at least two other active agents.

17-49. (canceled)

50. A method of treating a hepatitis C infection comprising administering to a patient in need thereof a MTP inhibitor in combination with at least one additional active agent, wherein the administration of the combination results in a greater reduction in the hepatitis C viral load of the patient as compared to administration of the additional active agent or the MTP inhibitor alone.

51. The method of claim 50, wherein the reduction in the hepatitis C viral load is greater than that achieved by administering the active agent alone or the MTP inhibitor alone.

52. The method of claim 50, wherein the reduction in the hepatitis C viral load is greater than the additive effect of administering the MTP inhibitor alone and the active agent alone.

53. The method of claim 50, wherein the viral load is measured using molecular nucleic acid testing methods.

54. The method of claim 50, wherein the viral load is measured at about 14 days, 28 days, 4 months and/or 6 months from a first administration.

55. The method of claim 50, wherein the additional active agent comprises a) interferon or pegylated interferon and b) ribavirin.

56. A method of treating hepatitis C comprising administering to a patient in need thereof a MTP inhibitor in combination with at least one additional active agent, wherein the administration of the combination results a shorter treatment duration as compared to administration of the additional active agent or the MTP inhibitor alone.

57-64. (canceled)
